(19) United States
(12) Patent Application Publication
(10) Pub. No.: US 2007/0166728 A1
(43) Pub. Date: Jul. 19, 2007

(54) GENETIC PROFILE IMAGING AND DATA-SHARING DEVICE AND METHODOLOGY FOR SOCIALLY RELEVANT TRAITS

(75) Inventor: Fredric David Abramson, Gaithersburg, MD (US)

Correspondence Address: Fredric Abramson, 9700 Great Seneca Highway, Rockville, MD, 20850, US

(73) Assignee: AlphaGenics, Inc., Rockville, MD (US)

(21) Appl. No.: 11/459,528

(22) Filed: Jul. 24, 2006

Related U.S. Application Data
(60) Provisional application No. 60/701,507, filed on Jul. 22, 2005.

(51) Int. Cl. C12Q 1/68; G06F 19/00

(52) U.S. Cl. 435/6; 702/20

(57) ABSTRACT

The present invention generally relates to genetic profiling and genetic data-sharing and, more particularly, is concerned with a system and method for genetic profile imaging and for determining and sharing information about a person's socially relevant traits. The present invention provides a means by which a person can safely share information about his or her genetic makeup, where the information is related to normal functions and unrelated to disease or illness.

### CROSS-REFERENCE TO RELATED APPLICATIONS

This application claims priority from U.S. Provisional Application No. 60/701,507, filed on Jul. 22, 2005, which is hereby incorporated by reference in its entirety.

### BACKGROUND OF THE INVENTION

1. Field of the Invention

The present invention generally relates to genetic profiling and genetic data-sharing and, more particularly, is concerned with a system and method for genetic profile imaging and for determining socially relevant traits that are part of a person's normal day-to-day functioning.

2. Description of the Related Art

People often associate historically, information about a person's genes has been associated with medical problems such as genetic diseases and predisposition toward a person discovering that she, he or a loved one has a gene for a bad disease. The basis of this negativity is understandable. For most of the period of genetic discovery in the ²⁰th Century, genes were identified as a result of some deviation typically discovered as deviations from normality. As early as the 1950's, scientists learned that extra or missing chromosomes correlated with severe handicaps such as Down Syndrome. Single gene disorders, known as Mendelian disorders, could be identified because these represented mutations in normal genes that are usually transmitted within a family. Disorders such as Sickle Cell Anemia, Phenylketonuria and Tay Sachs Disease became well known.

In the 1970's, it became possible to test for fetal genetic disorders. This test, called amniocentesis, was at first limited to looking at the fetus's chromosomes. Soon scientists developed other forms of prenatal biochemical and genetic testing. However, these procedures tended to foster a negative public perception of genetics because, in most cases, the reason a woman (or couple) undergoes prenatal testing is to consider the option of terminating the pregnancy. A decision to terminate because of a genetic defect can be traumatic.

Often, genes and genetics have a negative connotation. At the same time, people have an intense curiosity about who they are genetically. Knowing that the “fruit doesn't fall far from the tree” doesn't actually say much about who a person is. To satisfy this demand to learn more about ourselves, there is a need to create a positive aura around genetics, genes and genetic information by introducing an element of fun, enjoyment, and low risk social sharing.

Adults have high social needs. Many tend to congregate in public areas to meet potential dates and partners for and to start longer-term relationships. The social scene includes various means of ice breaking, often referred to as “lines.” Classic lines relate to a person's astrological sign, sports, physical appearance or food. Part of the social ritual is learning whether there is some possible compatibility, at least enough to justify spending time and energy in getting to know a person.

Most of the 30,000 genes in a person deal with normal functional processes. These include physical performance, creative thinking, musical ability and other normal activities. The identity of specific genes in a person and, as needed, the amount of activity of one or more of these genes can be performed using conventional methods such as high density microarrays, PCR, optical reading and biochemical testing. The tissues that can provide this information include saliva, cheek cells, blood, skin, hair and other tissues such as sperm that can be accessed in and/or obtained from a living person. In addition, certain genetic information can be obtained by various questionnaires, the answers to which can be reasonably correlated with specific portions of the respondent's underlying genetic makeup and genetic activity profile.

Consequently, a need exists for providing a means by which a person can share some harmless information, unrelated to health or illness, about the person's his/her normal genetic makeup, where the information relates to daily living and socially important issues and is unrelated to illness or disease. The present invention is directed to these and other needs.

### SUMMARY OF THE INVENTION

Additional aspects and/or advantages of the invention will be set forth in part in the description which follows and, in part, will be apparent from the description, or may be learned by practice of the invention.

The present invention is related to a system and method for genetic profile imaging and for determining socially relevant traits. The present invention is useful for a person to learn some information about his or her genetic makeup, where the information relates to lifestyle, preferences, routine behaviors and other socially useful and relevant traits and is unrelated to health or illness. The person may share some or all of the information as desired with third persons for a variety of purposes including determining compatibility.

Accordingly, the present invention relates to method for determining compatibility of a plurality of genetic data from a plurality of sources, wherein the genetic data are unrelated to disease or illness, which include the operative steps of: receiving a first genetic data from a first source and a second genetic data from a second source; comparing the first and second genetic data to a data source, wherein the data source comprises a plurality of socially relevant genes; determining whether the first and second genetic data comprise at least one of the plurality of socially relevant genes; displaying a first image corresponding to the first genetic data and a second image corresponding to the second genetic data; identifying a plurality of first and second identifiers from the first and second genetic data, wherein the first and second identifiers indicate a plurality of social traits; and comparing the first and second identifiers to determine whether the first and second source satisfy a predetermined genetic compatibility.

The method further includes the operative steps of: combining the compared first and second identifiers; transmitting the combined first and second identifiers to the second source; and generating a third image corresponding to the combined first and second identifiers. More specifically, the first and second identifiers are selected from a group comprising CTSD, IGF2R, CHRM2, MSX1 (HOX7), asp, BBNF, COMT, DRD4, DRD5, DRD2, DRD1, DRD3, MAOA, Fragile X, DLG1, DLG2, DLG3, DLG4, GRIA1, GRIN1, ApoE4, CREB1, CREB3, CREBBP, CREM, TORC2, TORC3, FGF, ARC or NOL3, EGR1 or zif268, CAMK2A, NR2B, Glur1, Homer, TR3 or NR4A1, Clacineurin inhibitor gene, Serotonin Transporter Genecystathionine- beta-synthasenerve growth factor receptors opsins , M opsin, L opsin, opn3, opn4, opn5, RHO, RGR, RRH, CBBM, CHML, CRX, GNAT1, GNAZ, GRK1, GRK7, CTSD, KMO, RANBP2, RBP3, RLBP1, VSX1, 11-cis-retinal , RDH12, SAG, PDE6B, Fork-head transcription factorOCP1, OCP2 (SKP1A), Oncomodulin, beta-parvalbumin ApoJ (CLU), ApoD, Octoconin90, Eya1, Cytochrome c oxidaseGJB2, GJB3, KCNQ1, KCNQ2, KCNQ3, KCNQ4, KCNQ5, KvLQT1, Isk, KCNE1, KCNE2, KCNE3, KCNE4, Slc2a2, GOOSCOID, RAR, RARA, RARB, RARG, Prx1 (PRRX1), Prx2 (PRRX2), Otx1, Otx2, Hoxal, Hoxbl, Fgf3, kreisler, Pax2, Hmx3, Brn-3c (POU4F3), NT3, BDNF, DFN1,2,4, DFNB9, DIAPH1, DIAPH2, Myol5, Myo7a, Prestin, ATQ1, FBXO2, Olfaction Receptors, ORs, RTP1, RTP2, RTP3, RTP4, REEP1, VN1R1, alpha-gustducin, Taste Receptorsalpha-transducingamma 13, Beta1, Beta3, retinal phosphodiesterase Trpm5, sac, PDE1A, PDE1B, PDE1C, PDE11a, T2R/TRB, T1R, T1R2, T1R3, T2R3, t2R10, PRH1, TRB7, RAX, Rx, CRX, Six1, Six2, Six4, Six6, Pitxl, Pitx2, Pitx3, PAX6, VSX1, Chx10, Prox1, Emx1, Emx2, EMX202, Msx1, Msx2, FOXG1B, DLX1, DLX2, DLX3, DLX5, Cut, ONECUT1, ONECUT2, ONECUT3, CUTL1, CUTL2, Gax, OTX1, OTX2, SOX9, VAX1, HOXA, HOXA1, HOXA2, HOXA5, HoxA10, HoxA11, HOXA11S, HOXA13, HoxA7, HoxA9, HOXB1, HoxB3, HoxB4, HoxC10, HOXD@, HoxD9, HoxD10, HOXC, HOXC6, HOXC8, HOXC9, HOXC10, HoxCl3, Cdx1, Cdx2, Cdx4, Hox11, Hox11L2, Six3/6, Esxr1, RHOX family, RHOX family, FOXP1, FOXP2, FOXP4, DRD4, DAT1, 5′-SLC6A4, VMAT2, HSR, Gene Name, 5-HTT (long form), 5-HTT (short form), MAO, D4DR (long form), D4DR (short form), AVPR1A, AGS3, Foxp2, CHRM2, IGF2R, CTSD, and VMAT2.

In one embodiment of the present invention, the method further includes the operative steps of: generating first and second coded pattern identifiers; associating the first and second coded pattern identifiers to the data source; and updating the data source to include the first and second coded pattern identifiers. In another embodiment, of the present invention, the method further includes the operative steps of identifying at least one of the plurality of socially relevant genes from a tissue source.

More specifically, the operative step of identifying at least one of the plurality of socially relevant genes comprises at least one of a plurality of techniques selected from a group comprising density microarrays, PCR, optical reading, biochemical testing, and questionnaires. In one embodiment, the tissue source for identifying at least one of the plurality of socially relevant genes is selected from a group comprising saliva, cheek cells, blood, skin, hair, and sperm. In another embodiment, the first and second identifiers comprise first and second gene maps. In yet another embodiment, the first and second images comprise holographic images of the first and second genetic data.

In one embodiment of the present invention, the method further includes the operative step of enabling a user to display plurality of images as a screen saver, said screen saver cycling through said plurality of images. In another embodiment of the present invention, the method further includes the operative step of masking a first portion of the first genetic data. More specifically, the step of masking a first portion of the first genetic data is performed by the first source.

The present invention also relates to a method for determining compatibility of a plurality of genetic data from at least one of a plurality of sources, wherein the genetic data are unrelated to disease or illness genotypes, which include the operative steps of: receiving a plurality of genetic data from at least one of the plurality of sources; determining whether the plurality of genetic data comprises at least one of the plurality of socially relevant genes; identifying a plurality of identifiers from the plurality of genetic data, wherein the identifiers indicate a plurality of social traits; comparing the identifiers to determine whether at least one of the plurality of sources satisfies a predetermined genetic compatibility; generating an image corresponding to the compared identifiers, wherein the compared identifiers corresponding to at least one of the plurality of sources satisfy a predetermined genetic compatibility; and transmitting the image to at least one of the plurality of compatible sources. In one embodiment of the present invention, the method further includes the operative steps of: determining proximity of at least one of the plurality of the compatible sources; and generating a signal to indicate the proximity of at least one of the plurality of the compatible source. In another embodiment of the present invention, the method further includes the operative step of generating a signal to indicate a level of genetic compatibility of at least one of the plurality of the compatible source.

The present invention is further related to a method for delivering data corresponding to a plurality of socially relevant traits to at least one of a plurality of sources, the socially relevant traits corresponding to a plurality of genetic data, wherein the genetic data are unrelated to disease or illness, which includes the operative steps of: determining whether the plurality of genetic data comprise at least one of a plurality of socially relevant traits; generating a signal corresponding to the plurality of socially relevant traits; and transmitting the signal to at least one of the plurality of sources. In one embodiment of the present invention, the method further includes the operative step of displaying an image corresponding to the transmitted signal.

The present invention provides a system for determining compatibility of a plurality of genetic data from at least one of a plurality of sources, wherein the genetic data are unrelated to disease or illness genotypes, which includes: a receiver for receiving a plurality of genetic data from at least one of the plurality of sources; a display unit for displaying at least one image corresponding to at least one of the plurality of the genetic data; a processor for determining whether at least one of the plurality of sources satisfies a predetermined genetic compatibility; an image processor for generating an image corresponding to at least one of the plurality of sources satisfying the predetermined genetic compatibility; and a transmitter for transmitting the image to at least one of the plurality of compatible sources. More specifically, the image processor is capable of generating holographic images of the genetic data.

In one embodiment, the system further includes: a sensor for determining proximity of at least one of the plurality of the compatible source; and a signal generator for generating a signal to indicate the proximity of at least one of the plurality of the compatible source. In another embodiment, the system further includes an amplifier for amplifying the signal generated by the signal generator to indicate a level of genetic compatibility of at least one of the plurality of the compatible source.

The present invention also provides a system for determining compatibility of a plurality of genetic data from at least one of a plurality of sources, wherein the genetic data are unrelated to disease or illness genotypes, which includes: a means for receiving a first genetic data from a first source and a second genetic data from a second source; a means for comparing the first and second genetic data to a data source, wherein the data source comprises a plurality of socially relevant genes; a means for determining whether the first and second genetic data comprises at least one of the plurality of socially relevant genes; a means for displaying a first image corresponding to the first genetic data and a second image corresponding to the second genetic data; a means for identifying a plurality of first and second identifiers from the first and second genetic data, wherein the first and second identifiers indicate a plurality of social traits; and a means for comparing the first and second identifiers to determine whether the first and second source satisfy a predetermined genetic compatibility.

In one embodiment, the system further includes: a means for generating a first and second coded pattern identifiers; a means for associating the first and second coded pattern identifiers to the data source; and a means for updating the data source to include the first and second coded pattern identifiers. In another embodiment, the system further includes the means for enabling a user to display plurality of images as a screen saver, said screen saver cycling through said plurality of images.

The present invention further provides a computer program product comprising a computer readable medium having computer readable code embodied therein, the computer readable code, when executed, causing a computer to implement a method for determining compatibility of a plurality of genetic data from at least one of a plurality of sources, wherein the genetic data are unrelated to disease or illness genotypes, the method includes the operative steps of: receiving a first genetic data from a first source and a second genetic data from a second source; comparing the first and second genetic data to a data source, wherein the data source comprises a plurality of socially relevant genes; determining whether the first and second genetic data comprise at least one of the plurality of socially relevant genes; displaying a first image corresponding to the first genetic data and a second image corresponding to the second genetic data; identifying a plurality of first and second identifiers from the first and second genetic data, wherein the first and second identifiers indicate a plurality of social traits; and comparing the first and second identifiers to determine whether the first and second source satisfy a predetermined genetic compatibility.

In one embodiment, the implemented method in the computer program product further includes the operative steps of: combining the compared first and second identifiers; transmitting the combined first and second identifiers to the second source; and generating a third image corresponding to the combined first and second identifiers.

In another embodiment, the implemented method in the computer program product further includes the operative steps of: generating a first and second coded pattern identifiers; and associating the first and second coded pattern identifiers to a data source, wherein the data source comprises a plurality of socially relevant genetic data.

The present invention also provides a computer program product comprising a computer readable medium having computer readable code embodied therein, the computer readable code, when executed, causing a computer to implement a method for determining compatibility of a plurality of genetic data from at least one of a plurality of sources, wherein the genetic data are unrelated to disease or illness genotypes, the method includes the operative steps of: receiving a plurality of genetic data from a plurality of sources; determining whether the plurality of genetic data comprises at least one of the plurality of socially relevant genes; identifying a plurality of identifiers from the plurality of genetic data, wherein the identifiers indicate a plurality of social traits; comparing the identifiers to determine whether at least one of the plurality of sources satisfies a predetermined genetic compatibility; generating an image corresponding to the compared identifiers, wherein the compared identifiers corresponding to at least one of the plurality of sources satisfy a predetermined genetic compatibility; and transmitting the image to at least one of the plurality of compatible sources.

In one embodiment, the implemented method in the computer program product further includes the operative steps of: determining proximity of at least one of the plurality of the compatible source; and generating a signal to indicate the proximity of at least one of the plurality of the compatible source. In another embodiment, the implemented method in the computer program product further includes the operative steps of generating a signal to indicate a level of genetic compatibility of at least one of the plurality of the compatible source.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is a schematic block diagram showing a system according to one embodiment of the present invention

### DETAILED DESCRIPTION OF THE PREFERRED EMBODIMENTS

Reference will now be made in detail to the embodiments of the present invention, examples of which are illustrated in the accompanying drawings, wherein like reference numerals refer to the like elements throughout. The embodiments are described below to explain the present invention by referring to the figures.

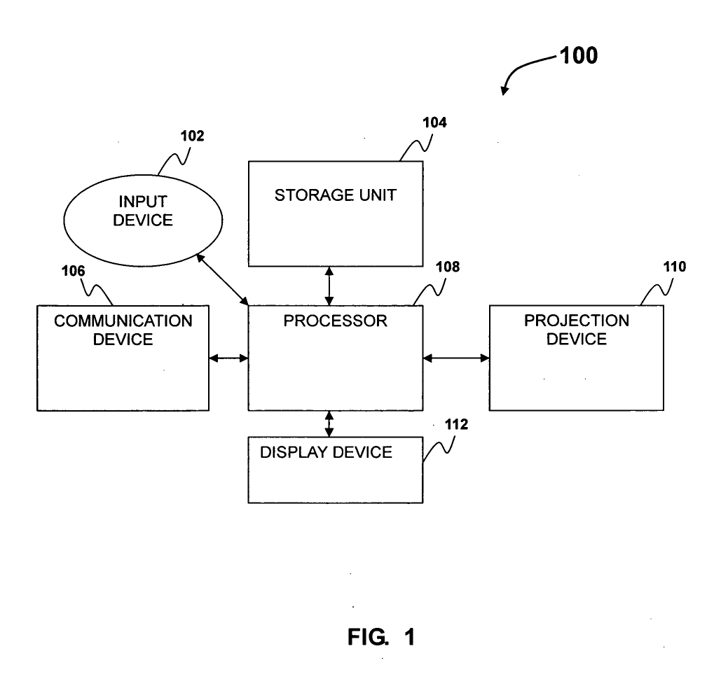

Referring generally to FIG. 1, there is shown a system for determining compatibility of genetic data from a plurality of sources, generally designated 100, which comprises the preferred embodiments of the present invention. System 100 includes an input device 102, a storage unit 104, a communication device 106, a processor 108, a projection device 110, and a display device 112.

The present invention introduces genes and genetics in an entirely new context: socialization. The present invention provides a means by which a person can share some information about his/her genetic makeup, where the information is essentially harmless and unrelated to disease or illness. The information will be shared in a variety of formats that take advantage of microelectronic packaging.

One embodiment of the present invention involves a handheld device about the size of a flip cell phone. The device electronics will permit electronic storage of information about the identity of a person's genes. The number of genes being stored will depend upon the user's desires and economics. More genes will command a higher price. The optimal number will be approximately 200 genes.

In one embodiment of the present invention, the system 100 will be capable of projecting a two-dimensional and/or a three-dimensional holographic image upon a nearby surface. In one example, the projection stream would be a laser system or a comparable mechanism that provides high visual coherence. The user would press a button to project the image. In another example, the image will be a graphic representation in 3-D, with a 2-D rendering, of the person's genes. It is expected that different polymorphs of a given gene will be identified by color, shape and/or size, or combinations of these. In yet another example, the graphic image would be dynamically changing in a fashion analogous to a screen saver.

System 100 will have one or more communications devices 106 that will be capable of sending and/or receiving data from another compatible device, or from the Internet or a cell phone. In one embodiment, this transfer of data may be by hard cable. In another embodiment, the transfer of data can be accomplished by one or more wireless protocols (e.g., Bluetooth™, 50-211).

Processor 108 will include at least one software program that will allow the user to (1) analyze a particular set of data for interesting patterns, (2) compare two or more sets of data, and (3) transmit the analysis and/or comparison to other devices, such as the display device 112 and projection device 110. Projection device 110 will project graphic images of two or more combined and compared data sets. In one embodiment, the visual comparison would include specially coded pattern identifiers. Each specially coded identifier will be linked to a compatibility manual that can be used by the user to explore whether two persons with an identifier are potentially socially compatible.

It will further be appreciated by those skilled in the art that the term “genotype” refers to the particular genetic pattern seen in the DNA of an individual. Thus the overall aesthetics of both the device and the graphics will be such that young adults would be attracted to them while sustaining interest and support from older adults. Another aspect of the device design is usually used to refer to the particular pair of alleles that an individual possesses at a certain location in the genome, and can be considered the entire genetic identity of an individual, including alleles. Holding a device is a sensual experience. The more a person enjoys holding it, the more likely they are to use it. The more use that is made of it, the more likely other people in the same social setting will want to use it too.

In one embodiment of the present invention, the genes that are chosen may include the attributes that existed in the past or gene forms that do not show as outward characteristics. It will also be appreciated by those skilled may exist in the art that the amount of genetic activity future, or gene expression, can be measured for the genes that comprise the person's genotype.

One aspect of the device concerns its look and feel. Young adults are attracted to stylish or popular devices with a high “cool” factor such as the iPod™. Thus the overall aesthetics of both the device and the graphics will be such that young adults would be attracted to them. Another aspect of the device is tactile appeal. Holding a device is a sensual experience. The more a person enjoys holding it, the more likely he or she is to use it. The more use that is made of it, the more likely other people in the same social setting will want to use it too.

In one embodiment of the present invention, the genes that are chosen may include the following attributes: (1) they either have no known biological function or have a function that is irrelevant for an adult (examples that may change over time. Examples of such genes might include hair color, the rate of hair growth, eruption of the first tooth as an infant, and so on), (2) they are highly polymorphic in the population, which means there are a variety of different genes in circulation.

The point of relating to normal functioning is essential to the invention. Targeting “disease” genes would defeat the purpose of the invention, which is to make genes and genetics a ‘fun’ aspect of a person's social world. Learning a person has a gene for, say, cystic fibrosis or breast cancer could powerfully interfere with social functions.

There is also the issue of privacy, as a person's health future from a genetic point of view is highly private. So selecting “relevant” genes would mean that the data must be protected so that no third person can access the data if the device falls into their hands.

The point of high variability in the population relates to the fact that greater differences between people provides more to talk about. If everyone has the same gene, there's no reason to share the information.

The present invention has many different benefits to fulfill a variety of social relationships. One use is to let people, such as singles, family members, etc., compare their visual gene maps. Each person can project a holographic image of their three dimensional genetic portrait. If projected onto a solid surface, a two dimensional portrait is displayed. Although the general map structure will be the same for all holograms, specific genetic attributes will be differentiated in a variety of ways. These ways include color, shapes, size and so on.

One alternative to visual comparison would be a computational comparison. In one embodiment, the present invention may incorporate as optional or standard feature communications devices 106 as well as with other electronic devices such as cell phones, PDA's and computers. The communication modality will be Blue Tooth or other standard protocol.

The present invention can include compatibility software which will compare different gene profiles.

In some embodiments of the present invention, data can also be uploaded to a computing device for Internet use or other use. Users of the present invention may be able to share data with other users of the present invention located at any place around the world. The present invention may be used to create SkyGene web sites to facilitate development of a community of users.

In one embodiment of the present invention, one or more images of the users may be projected along with the map design. This feature can have particular benefit for Internet use because the receiving user can see a projection not only of the person's genetic map but also a portrait.

In other embodiments, the present invention can be used for educational purposes. Teachers can use the present invention to illustrate genetic variation as well as other features of genetics.

In yet another embodiment, the present invention can be used as a lost-child identifier. Storage unit 104 includes a person's unique genetic makeup for as many as 200 or more genes. The present invention can be used to identify children whose identities may have been changed, as for example when an estranged parent removes the child without permission.

Embodiments of the present invention may include traits that are shaped or influenced by more than one gene may have some or all of the known genes included in the profile. Exemplary genes classes include IQ, Intelligence, Ambition, Creativity, Mathematical ability, Foreign Language ability, Language ability, Aggressiveness, Possessiveness, Sensuality, Sense of humor, Kindness, Self Control, Sexual Desire, Temper, Jealousy, Optimism, Pessimism, Caring, Impatience , Anger, Passivity, Hair Color, Allergies, Eye color, Height, Toenail growth, Foot size, Body type, Sexual orientation, Lustfulness, Ancestry, Musical ability, Dance, Artistic ability, Movement, Drug use, Obsession, Gambling, Alcohol consumption and tolerance, Affinity for Smoking, Compulsive behavior, Sexual endurance, Athletic endurance, Mental endurance, Pain endurance, Pheromones, Olfaction, Vision, Fetishism, Taste, Hearing, Affinity for Chocolate, Wine Tasting ability, Introversion, Extroversion, Communication, Listening, Empathy, Spirituality, Leadership, Boldness, and the like. Accompanying the genes selected may be one or more questionnaires, biochemical or other testing, where the results of such questionnaires or tests help explain or otherwise add information about the gene or the trait.

In one embodiment of the present invention, the software that lets the user select desired genetic profiles in other people is programmable. A system embodying the present invention reads other devices within communication distance and identifies how closely the sending person's profile matches the user's desires. As the profile being received comes close to the desired profile, the device begins to vibrate, where the intensity of the vibration is related to the closeness of the profile and the physical proximity of the user to the person.

The present invention includes display capabilities. In one embodiment, the display is a 3-D holographic projection system that uses a pre-printed holographic film and a laser or other light source. The 3-D hologram can be projected into the air, or onto a surface with a two dimensional effect. Another holographic display uses a layer of thin films where each film layer has a portion of the final image. The image on each film layer is generated in digital form using a variety of computing and transmission means. The passing of a light beam through the film layers from a light source produces a 3-D holographic effect. In another embodiment, the display means may be a liquid crystal display or other display technologies used in cell phones and laptop computers.

In some embodiments, core numbers of genes that are common to all users are projected in the 3-D hologram or other image projections. Additional genes are identified and provided to the user with levels of encoding which the user can change at will. The encoding levels provide a means by which the user may choose to reveal or not reveal the identity of any gene to any particular person.

In one example of the present invention, 3-D and 2-D image projections may be defined and modified by the genes which the person chooses to reveal. In another example, the present invention incorporates into the displayed image of genes the image or photograph of the user whose genes are being displayed. This provides a means of identifying the person. In yet another example, the present invention provides the user the identity and activity of certain genes in a child, with the purpose being to enable the adult to better understand aspects of the child's potential in physical and mental activities, as an aid to better planning the child's future.

Communications between the present invention and other devices, including other embodiments of the present invention, may be by wireless means (e.g., Bluetooth™), cable connection and USB connections, and the like.

According to the present invention, software will permit the user to compare profiles. The comparisons can be weighted according to pre-determined criteria or by criteria entered by the user. The user can compare his or her profile with any other person or persons, or compare two or more other profiles. The extent to which compared profiles match or fail to match can be scored and ranked on numerical or non-numeric scales or both as desired by the user.

Comparison results can be communicated to any other device, including output devices, and displayed on the display device 112 in tabular, graphic or image form. For image comparisons, the present invention may be used to formulate an image that represents and portrays the degree of match or non-match by changing all or portions of the image. The changes may involve shape, position and/or color, as well as intensity and shading, as well as any other way which communicates to the user where there is overlap and where there is non-overlap, as well as the option for the degree of overlap. In one embodiment, the image can be zoomed as well as segmented, so that only a portion of the image is displayed. This zoom and segmentation can be controlled by the user as well as pre-programmed.

Data received and transmitted by communication device 106 are protected by passwords and one or more encryption methods so that the data cannot be taken or used by third persons who may acquire the device, such as when the device is lost or stolen.

Table 1 illustrates exemplary candidate genes that may be included in some embodiments of the present invention.

TABLE 1

| Gene Name | Trait | Relevance |
|---|---|---|
| CTSD | Cognition | Lysosomal proteinases. Deletions in animal models led to |
| | | decreased brain activity |
| IGF2R | Cognition | Was linked to high intelligence but replicates were not |
| | | positive. This lead to a 2nd IQ related polymorphism in the |
| | | IGF2R gene |
| CHRM2 | Cognition | cholinergic receptor |
| MSX1 (HOX7) | Cognition | a homeobox gene important for brain development |
| asp | Cognition | the size of asp parallels brain size across several species |
| BDNF | Cognition | Associated with impaired performance of memory tasks |
| COMT | Cognition | Influences the activation of working memory circuits. COMT |
| | | polymorphisms seem to be highly specific to some |
| | | prefrontal cortex-dependent tasks in children. |
| DRD4 | Cognition | Polymorphisms are associated with differences in |
| | | performance and brain activity during tasks that involve |
| | | executive attention. |
| DRD5 | | |
| DRD2 | | |
| DRD1 | | |
| DRD3 | | |
| MAOA | Cognition | Polymorphs associated with difference related to executive |
| | | attention |
| Fragile X | Cognition | About 70 genes on X chromosome might be responsible for |
| | | learning disabilities. Also suppressors of Fragile X |
| DLG1 | | |
| DLG2 | | |
| DLG3 | Cognition | Mutation in gene linked to learning disability. SAP102 |
| | | (which is related to PSD95 or postsynaptic density 95) is |
| | | DLG3 protein product and binds to NMDA receptor. |
| DLG4 | | |
| GRIA1 | | |
| GRIN1 | | |
| ApoE4 | Cognition | |
| CREB1 | Cognition | This is a 2nd messenger pathway member that is related to |
| | | memory. It might be worthwhile to investigate variations in |
| | | signaling cascades. If we can discover polymorphisms in |
| | | these second messengers then perhaps that will account for |
| | | some of the variations we see in individual traits. Signaling |
| | | cascades are crucial in sensory input. |
| CREB3 | | |
| CREBBP | Cognition | |
| CREM | | |
| TORC2 | Cognition | |
| TORC3 | Cognition | |
| FGF | Cognition | FGFs play imp roles in embryonic development, cell growth, |
| | | morphogenesis, tissue repair, inflammation, angiogenesis, |
| | | and tumor growth and invasion. |
| ARC or NOL3 | Cognition | apoptosis repressor |
| EGR1 or zif268 | Cognition | Early growth response 1 |
| CAMK2A | Cognition | calcium/calmodulin-dependent protein kinase II-alpha. |
| | | Abundant in the brain as a major constituent of the |
| | | postsynaptic density |
| NR2B | Cognition | Glutamate receptor, ionotrophic, N-Methyl-D-Aspartate. |
| | | NMDA receptor channel has been shown to be involved in |
| | | long-term potentiation, an activity-dependent increase in the |
| | | efficiency of synaptic transmission thought to underlie |
| | | certain kinds of memory and learning. |
| Glur1 | Cognition | Glutamate receptor. GluR are the predominant excitatory |
| | | neurotransmitter receptors in the mammalian brain and are |
| | | activated in a VARIETY of normal neurophysiologic |
| | | processes. |
| Homer | Cognition | Homer is a neuronal immediated-early gene that is enriched |
| | | at excitatory synapses and binds mGluRs |
| TR3 or NR4A1 | Cognition | Nuclear hormone receptor TR3 |
| Clacineurin inhibitor | Cognition | helps in learning and memory |
| gene | | |
| Serotonin | cystathionine-beta- | nerve growth factor receptor |
| Transporter Gene | synthase | |
| S opsins | Vision | Night vision: bat S opsins indicate sensitivity to |
| | | UV.Circadian rhythms may be regulated by opsins. Also |
| | | might be related to Drosophilia Per and Tim interplay. |
| M opsin | Vision | M and L opsin genes can be duplicated or deleted (color |
| | | blindness) and the number of copies may produce a |
| | | measurable variance in the population. |
| L opsin | Vision | M and L opsin genes can be duplicated or deleted (color |
| | | blindness) and the number of copies may produce a |
| | | measurable variance in the population. |
| opn3 | Vision | |
| opn4 | Vision | |
| opn5 | Vision | |
| RHO | Vision | |
| RGR | Vision | |
| RRH | Vision | |
| CBBM | Vision | |
| CHML | Vision | |
| CRX | Vision | |
| GNAT1 | Vision | |
| GNAZ | Vision | |
| GRK1 | Vision | |
| GRK7 | Vision | |
| CTSD | Vision | |
| KMO | Vision | |
| RANBP2 | Vision | |
| RBP3 | Vision | |
| RLBP1 | Vision | |
| VSX1 | Vision | |
| 11-cis-retinal | Vision | Chromophore that plays a role in color vision. As do |
| | | transducin and cGMP-K+ selective channels. |
| RDH12 | | |
| SAG | | |
| PDE6B | | |
| Fork-head | | |
| transcription factor | | |
| OCP1 | Audition | OCPs are proteins abundant in the supporting cells of the |
| | | organ of Corti, the sensory organ of the inner ear |
| OCP2 (SKP1A) | Audition | OCPs are proteins abundant in the supporting cells of the |
| | | organ of Corti, the sensory organ of the inner ear |
| Oncomodulin | Audition | a novel Ca2+ binding protein of the beta-parvalbumin |
| | | sublineage, exclusively expressed in the outer hair cells of |
| | | the organ of corti |
| beta-parvalbumin | | |
| ApoJ (CLU) | Audition | Apo's proteins highly concentrated in the inner ear fluids, |
| | | endolymph, and perilymph. |
| ApoD | Audition | Apo's proteins highly concentrated in the inner ear fluids, |
| | | endolymph, and perilymph. |
| Octoconin90 | Audition | Major mammalian protein of the otoconia, a complex of |
| | | proteins and minerals that sit on top of the vestibular gravity |
| | | receptor organ. |
| Eya1 | Audition | Has a role in development of all components of the inner |
| | | ear. |
| Cytochrome c oxidase | | |
| GJB2 | Audition | Connexin 26 |
| GJB3 | Audition | connexin 31 |
| KCNQ1 | | |
| KCNQ2 | | |
| KCNQ3 | | |
| KCNQ4 | Audition | POTASSIUM CHANNEL, VOLTAGE-GATED, KQT-LIKE |
| | | SUBFAMILY |
| KCNQ5 | | |
| KvLQT1 | Audition | POTASSIUM CHANNEL, VOLTAGE-GATED, KQT-LIKE |
| | | SUBFAMILY, MEMBER 1 |
| Isk | Audition | POTASSIUM CHANNEL, VOLTAGE-GATED, ISK- |
| | | RELATED SUBFAMILY, MEMBER 1; KCNE1 |
| KCNE1 | | |
| KCNE2 | | |
| KCNE3 | | |
| KCNE4 | | |
| Slc2a2 | Audition | SOLUTE CARRIER FAMILY 2 (FACILITATED GLUCOSE |
| | | TRANSPORTER), MEMBER 2; SLC2A2 |
| GOOSCOID | Audition | |
| RAR | Audition | RETINOIC ACID RECEPTOR |
| RARA | | |
| RARB | | |
| RARG | | |
| Prx1 (PRRX1) | Audition | PAIRED-RELATED HOMEOBOX GENE 1 |
| Prx2 (PRRX2) | Audition | PAIRED-RELATED HOMEOBOX GENE 2 |
| Otx1 | Audition | ORTHODENTICLE, DROSOPHILA, HOMOLOG OF, 1 |
| Otx2 | Audition | OTX2 is a homeobox family gene related to a Drosophila |
| | | gene expressed in the developing head |
| Hoxa1 | Audition | |
| Hoxb1 | Audition | |
| Fgf3 | Audition | |
| kreisler | Audition | |
| Pax2 | Audition | PAIRED BOX GENE 2 |
| Hmx3 | Audition | |
| Brn-3c (POU4F3) | Audition | POU DOMAIN, CLASS 4, TRANSCRIPTION FACTOR 3 |
| NT3 | Audition | NEUROTROPHIC FACTOR 3 |
| BDNF | Audition | |
| DFN1,2,4 | Audition | |
| DFNB9 | Audition | |
| DIAPH1 | Audition | |
| DIAPH2 | | |
| Myo15 | Audition | |
| Myo7a | Audition | |
| Prestin | Audition | Prestin is the motor protein of cochlear outer hair cells. |
| ATQ1 | Audition | |
| FBXO2 | | |
| Olfaction Receptors | Olfaction | full length sequences of 347 hORs |
| ORs | Olfaction | Canine and chimp olf. Subgenomes and their human |
| | | counterparts |
| RTP1 | Olfaction | receptor transporting proteins 1&2 that are expressed in |
| | | olfactory and vomeronasal organs |
| RTP2 | Olfaction | receptor transporting proteins 1&2 that are expressed in |
| | | olfactory and vomeronasal organs |
| RTP3 | Olfaction | |
| RTP4 | Olfaction | |
| REEP1 | Olfaction | Receptor expression-enhancing protein 1 promotes |
| | | functional cell surface expression of ORs. REEP1 was |
| | | associated with OR proteins and enhanced the OR |
| | | responses to odorants. |
| VN1R1 | Pheromonal | vomeronasal 1 receptor 1 |
| alpha-gustducin | Taste | |
| Taste Receptors | | |
| alpha-transducin | | |
| gamma 13 | Taste | |
| Beta1 | Taste | |
| Beta3 | Taste | |
| retinal phosphodiesterase | | |
| Trpm5 | Taste | ion channel |
| sac | Taste | |
| PDE1A | Taste | |
| PDE1B | | |
| PDE1C | | |
| PDE11a | Taste | |
| T2R/TRB | Taste | |
| T1R | Taste | |
| T1R2 | Taste | |
| T1R3 | Taste | |
| T2R3 | Taste | |
| T2R10 | Taste | |
| PRH1 | Taste | |
| TRB7 | Taste | |
| | Taste | Taste Sensitivity SNP |
| | Taste | Taste Sensitivity SNP |
| RAX | | retina and anterior neural fold homeobox |
| Rx | Homeobox | controls proliferation and neurogenesis in anterior neural |
| | | plate and of retinal progenitors (targets for brain size and |
| | | eye formation, maybe leading to differences in sight) |
| CRX | | cone-rod homeobox |
| Six1 | Homeobox | Expressed during MOUSE development in otic vesicles, |
| | | nasal epithelia, branchial arches/pouches, nephrogenic |
| | | cords, somites and a limited set of ganglia. Six1-deficient |
| | | mice display sever abnormalities in the formation of the |
| | | inner ear, thymus, nose, kidney and skeletal muscle |
| Six2 | | |
| Six4 | | |
| Six6 | Homeobox | involved in eye development |
| Pitx1 | | |
| Pitx2 | Homeobox | Involved in pituitary development. The pituitary is directed |
| | | to send out signals concerning growth, eating, drinking, and |
| | | maternal behavior. Also involved in the motivational system |
| | | of the brain, initiation and maintaining behaviors the |
| | | individual finds rewarding. |
| Pitx3 | | |
| PAX6 | | |
| VSX1 | | |
| Chx10 | Homeobox | exclusively expressed in proliferatively active neuroblasts of |
| | | the neuroretina starting from optic cup formation |
| Prox1 | Homeobox | Has a negative role on retinal progenitor cell proliferation. |
| Emx1 | | |
| Emx2 | Homeobox | required for the formation of hippocampus of proper size but |
| | | not for its specification nor its patterning |
| EMX2O2 | | |
| Msx1 | Homeobox | Expressed in the developing and differentiated tissues of |
| | | the mammary gland. |
| Msx2 | Homeobox | Expressed in the developing and differentiated tissues of |
| | | the mammary gland. |
| FOXG1B | | |
| DLX1 | | |
| DLX2 | | |
| DLX3 | | |
| DLX5 | | |
| Cut | Homeobox | Cut proteins are expressed in the brain, limb, lung and |
| | | kidney are act as transcriptional repressors |
| ONECUT1 | | |
| ONECUT2 | | |
| ONECUT3 | | |
| CUTL1 | | |
| CUTL2 | | |
| Gax | Homeobox | A homeobox gene that is expressed in adult vascular |
| | | smooth muscular cells and embryonic cardiac, smooth and |
| | | skeletal muscle. It responds to vascular injury, correlating |
| | | cell proliferation to repair the tissue |
| OTX1 | | |
| OTX2 | | |
| SOX9 | | |
| VAX1 | | |
| HOXA | | homeobox A cluster |
| HOXA1 | | |
| HOXA2 | | |
| HOXA5 | | |
| HoxA10 | Homeobox | |
| HoxA11 | Homeobox | |
| HOXA11S | | |
| HOXA13 | | |
| HoxA7 | Homeobox | |
| HoxA9 | Homeobox | |
| HOXB1 | | |
| HoxB3 | | |
| HoxB4 | Homeobox | |
| HoxC10 | Homeobox | |
| HOXD@ | | |
| HoxD9 | | |
| HoxD10 | Homeobox | |
| HOXC | | |
| HOXC6 | | |
| HOXC8 | | |
| HOXC9 | | |
| HOXC10 | | |
| HoxC13 | Homeobox | |
| Cdx1 | Homeobox | play crucial roles in gut development and homeostasis |
| Cdx2 | Homeobox | play crucial roles in gut development and homeostasis |
| Cdx4 | | |
| Hox11 | Homeobox | Required for spleen development and has also been |
| | | implicated in human t-cell leukemia. |
| Hox11L2 | Homeobox | |
| Six3/6 | Homeobox | reported to influence the proliferation of retinal progenitor |
| | | cells |
| Esxr1 | Homeobox | mainly expressed in placenta and testis |
| RHOX family | Homeobox | RHOX family of homeobox transcription factors (RHOX1-11) |
| | | are expressed in reproductive tissue. In the testis they are |
| | | involved in sperm count, sperm motility, apoptosis in |
| | | spermatocytes, spermatogonia differentiation, spermatid |
| | | maturation. In other words sexual potency. |
| RHOX family | Homeobox | |
| FOXP1 | | |
| FOXP2 | Creativity | Supposedly the first gene linked conclusively to language, |
| | | and began our cultural revolution. |
| FOXP4 | | |
| DRD4 | Creativity | a dopamine-receptor |
| DAT1 | Creativity | dopamine-transporter gene |
| 5′-SLC6A4 | Creativity | Preliminary findings suggest that a polymorphism in the 5′ |
| | | promoter region (5-HTTLPR) of the 5′-SLC6A4 gene is |
| | | significantly associated with and contributes to the variance |
| | | of the creativity index and the temperament and character |
| | | profile.” |
| VMAT2 | Creativity/God gene | Vesicular monoamine transporter (VMAT2) had a strong |
| | | correlation with religiosity (God gene) and seems to have a |
| | | role in BD, and therefore, possibly has a role in creativity. |
| HSR | Behaviors | Hand skill, relative (handedness) |
| | Behaviors | Hand clasping pattern |
| | Behaviors | HAIR WHORL |
| | Behaviors | Arm folding preference |
| | Behaviors | Ears, ability to move |
| | Behaviors | Tongue curling, folding, or rolling |
| | Behaviors | Musical perfect pitch |
| | Behaviors | Tune Deafness |
| | Behaviors | Novelty seeking personality trait |
| | Behaviors | Novelty seeking personality trait |
| | Behaviors | Stuttering |
| | Behaviors | Stuttering |
| | Behaviors | Tobacco addiction |
| | Behaviors | Tobacco addiction |
| | Behaviors | Tobacco addiction |
| | Behaviors | Tobacco addiction |
| | Behaviors | Alcoholism |
| | Behaviors | Alcoholism |
| | Behaviors | Alcoholism |
| | Behaviors | Alcoholism |
| | Behaviors | Alcoholism |
| | Behaviors | Obsessive-Compulsive Disorder |
| | Behaviors | Homosexuality |
| | Behaviors | Homosexuality |
| | Behaviors | Homosexuality |
| | Behaviors | Monogamy/Fidelity |
| | Behaviors | Monogamy/Fidelity |
| | Behaviors | Monogamy/Fidelity |
| | Behaviors | Social Behavior |
| | Behaviors | Social Behavior |
| | Behaviors | Social Behavior |
| | Behaviors | Social Behavior |
| Gene Name | Regulates | Category (Artistic, et.) |
| 5-HTT (long form) | extravert | Social |
| 5-HTT (short form) | Introvert | Social |
| MAO | Aggressive | Social |
| D4DR (long form) | extravert | Social |
| D4DR (short form) | Introvert | Social |
| AVPR1A | Increase social | |
| | behavior | |
| AGS3 | pleasure | |
| Foxp2 | communication | |
| CHRM2 | cognitive process | social |
| IGF2R | cognitive process | |
| CTSD | cognitive process | |
| VMAT2 | spirituality | GOD gene |

Although a few embodiments of the present invention have been shown and described, it would be appreciated by those skilled in the art that changes may be made in these embodiments without departing from the principles and spirit of the invention, the scope of which is defined in the claims and their equivalents.

It will further be appreciated by those skilled in the art that the term “genotype” refers to the particular genetic pattern seen in the DNA of an individual. “Genotype” is usually used to refer to the particular pair of alleles that an individual possesses at a certain location in the genome, and can be considered the entire genetic identity of an individual, including alleles, or gene forms, that do not show as outward characteristics.

It will also be appreciated by those skilled in the art that the amount of genetic activity, or gene expression, can be measured for the genes that comprise the person's genotype.

What is claimed is:

1. A method for sharing information of a plurality of genetic data from a plurality of sources, wherein the genetic data are unrelated to disease or illness, comprising:
receiving a first genotypic data from a first source and a second genotypic data from a second source;
comparing the first and second genotypic data to a data source, wherein the data source comprises a plurality of socially relevant genes;
determining whether the first and second genotypic data comprises at least one of the plurality of socially relevant genes;
displaying a first image corresponding to the first genetic data and a second image corresponding to the second genotypic data;
identifying a plurality of first and second identifiers from the first and second genotypic data, wherein the first and second identifiers indicate a plurality of social traits; and
comparing the first and second identifiers to determine whether the first and second source satisfy a predetermined genetic compatibility for one or more criteria relating to social compatibility,
wherein the first and second genotypic data is related to normal functions and unrelated to disease or illness.

2. The method of claim 1, further comprising:
combining the compared first and second identifiers;
transmitting the combined first and second identifiers to the second source; and
generating a third image corresponding to the combined first and second identifiers.

3. The method of claim 1, further comprising:
generating a first and second coded pattern identifiers;
associating the first and second coded pattern identifiers to the data source; and
updating the data source to include the first and second coded pattern identifiers.

4. The method of claim 1, further comprising identifying at least one of the plurality of socially relevant genes from a tissue source.

5. The method of claim 4, wherein the step of identifying at least one of the plurality of socially relevant genes comprises at least one of a plurality of techniques selected from a group comprising density microarrays, PCR, optical reading, biochemical testing, and questionnaires.

6. The method of claim 4, wherein the tissue source for identifying at least one of the plurality of socially relevant genes is selected from a group comprising saliva, cheek cells, blood, skin, hair, and sperm.

7. The method of claim 1, wherein the first and second identifiers comprise first and second gene maps.

8. The method of claim 1, wherein the first and second images comprise holographic images of the first and second genetic data.

9. The method of claim 1, further comprising the step of enabling a user to display plurality of images as a screen saver, said screen saver cycling through said plurality of images.

10. The method of claim 1, wherein the first and second identifiers are selected from a group comprising CTSD, IGF2R, CHRM2, MSX1 (HOX7), asp, BDNF, COMT, DRD4, DRD5, DRD2, DRD1, DRD3, MAOA, Fragile X, DLG1, DLG2, DLG3, DLG4, GRIA1, GRIN1, ApoE4, CREB1, CREB3, CREBBP, CREM, TORC2, TORC3, FGF, ARC or NOL3, EGR1 or zif268, CAMK2A, NR2B, Glur1, Homer, TR3 or NR4A1, Clacineurin inhibitor gene, Serotonin Transporter Genecystathionine-beta-synthasenerve growth factor receptors opsins, M opsin, L opsin, opn3, opn4, opn5, RHO, RGR, RRH, CBBM, CHML, CRX, GNAT1, GNAZ, GRK1, GRK7, CTSD, KMO, RANBP2, RBP3, RLBP1, VSX1, 11-cis-retinal, RDH12, SAG, PDE6B, Fork-head transcription factorOCP1, OCP2 (SKP1A), Oncomodulin, beta-parvalbumin ApoJ (CLU), ApoD, Octoconin90, Eyal, Cytochrome c oxidaseGJB2, GJB3, KCNQ1, KCNQ2, KCNQ3, KCNQ4, KCNQ5, KvLQT1, Isk, KCNE1, KCNE2, KCNE3, KCNE4, Sic2a2, GOOSCOID, RAR, RARA, RARB, RARG, Prx1 (PRRX1), Prx2 (PRRX2), Otx1, Otx2, Hoxa1, Hox1, Fgf3, kreisler, Pax2, Hmx3, Brn-3c (POU4F3), NT3, BDNF, DFN1,2,4, DFNB9, DIAPH1, DIAPH2, Myol5, Myo7a, Prestin, ATQ1, FBXO2, Olfaction Receptors, ORs, RTP1, RTP2, RTP3, RTP4, REEP1, VN1 R1, alpha-gustducin, Taste Receptorsalpha-transducingamma 13, Beta1, Beta3, retinal phosphodiesterase Trpm5, sac, PDE1A, PDE1B, PDE1C, PDE11a, T2R/TRB, T1R, T1R2, T1R3, T2R3, T2R10, PRH1, TRB7, RAX, Rx, CRX, Six1, Six2, Six4, Six6, Pitx1, Pitx2, Pitx3, PAX6, VSX1, Chx10, Prox1, Emx1, Emx2, EMX202, Msx1, Msx2, FOXG1 B, DLX1, DLX2, DLX3, DLX5, Cut, ONECUT1, ONECUT2, ONECUT3, CUTL1, CUTL2, Gax, OTX1, OTX2, SOX9, VAX1, HOXA, HOXA1, HOXA2, HOXA5, HoxA10, HoxA11, HOXAL11S, HOXA13, HoxA7, HoxA9, HOXB1, HoxB3, HoxB4, HoxCl0, HOXD@, HoxD9, HoxD10, HOXC, HOXC6, HOXC8, HOXC9, HOXC10, HoxC13, Cdx1, Cdx2, Cdx4, Hox11, Hox11L2, Six3/6, Esxr1, RHOX family, RHOX family, FOXP1, FOXP2, FOXP4, DRD4, DAT1,5′-SLC6A4, VMAT2, HSR, Gene Name, 5-HTT (long form), 5-HTT (short form), MAO, D4DR (long form), D4DR (short form), AVPR1A, AGS3, Foxp2, CHRM2, IGF2R, CTSD, and VMAT2.

11. The method of claim 1, further comprising masking a first portion of the first genetic data.

12. The method of claim 11, wherein the step of masking a first portion of the first genetic data are performed by the first source.

13. A method for determining compatibility of a plurality of genetic data from at least one of a plurality of sources, wherein the genetic data are unrelated to disease or illness genotypes, comprising:
receiving a plurality of genetic data from at least one of the plurality of sources;
determining whether the plurality of genetic data comprises at least one of the plurality of socially relevant genes;
identifying a plurality of identifiers from the plurality of genetic data, wherein the identifiers indicate a plurality of social traits;
comparing the identifiers to determine whether at least one of the plurality of sources satisfies a predetermined genetic compatibility;
generating an image corresponding to the compared identifiers, wherein the compared identifiers corresponding to at least one of the plurality of sources satisfy one or more criteria relating to social compatibility; and
transmitting the image to at least one of the plurality of compatible sources,
wherein the plurality of genetic data is related to normal functions and unrelated to health or illness.

14. The method of claim 13, further comprising:
determining proximity of at least one of the plurality of the compatible sources; and
generating a signal to indicate the proximity of at least one of the plurality of the compatible source.

15. The method of claim 13, further comprising generating a signal to indicate a level of genetic compatibility of at least one of the plurality of the compatible source.

16. A method for delivering data corresponding to a plurality of socially relevant traits to at least one of a plurality of sources, the socially relevant traits corresponding to a plurality of genetic data, wherein the genetic data are unrelated to disease or illness, comprising:
determining whether the plurality of genetic data comprise at least one of a plurality of socially relevant traits;
generating a signal corresponding to the plurality of socially relevant traits; and
transmitting the signal to at least one of the plurality of sources.

17. The method of claim 16, further comprising displaying an image corresponding to the transmitted signal.

18. A system for determining compatibility of a plurality of genetic data from at least one of a plurality of sources, wherein the genetic data are unrelated to disease or illness genotypes, comprising:
a receiver for receiving a plurality of genetic data from at least one of the plurality of sources;
a display unit for displaying at least one image corresponding to at least one of the plurality of the genetic data;
a processor for determining whether at least one of the plurality of sources satisfy a predetermined genetic compatibility;
an image processor for generating an image corresponding to at least one of the plurality of sources relating to at least one criterion relating to social compatibility; and
a transmitter for transmitting the image to at least one of the plurality of compatible sources,
wherein the genetic data is related to normal functions and is unrelated to disease or illness.

19. The system of claim 18, further comprising:
a sensor for determining proximity of at least one of the plurality of the compatible source; and
a signal generator for generating a signal to indicate the proximity of at least one of the plurality of the compatible source.

20. The system of claim 17, further comprising an amplifier for amplifying the signal generated by the signal generator to indicate a level of genetic compatibility of at least one of the plurality of the compatible source.

21. The system of claim 18, wherein the image processor is capable of generating holographic images of the genetic data.

22. A system for determining compatibility of a plurality of genetic data from at least one of a plurality of sources, wherein the genetic data are unrelated to disease or illness genotypes, comprising:
a means for receiving a first genetic data from a first source and a second genetic data from a second source;
a means for comparing the first and second genetic data to a data source, wherein the data source comprises a plurality of socially relevant genes;
a means for determining whether the first and second genetic data comprises at least one of the plurality of socially relevant genes;
a means for displaying a first image corresponding to the first genetic data and a second image corresponding to the second genetic data;
a means for identifying a plurality of first and second identifiers from the first and second genetic data, wherein the first and second identifiers indicate a plurality of social traits; and
a means for comparing the first and second identifiers to determine whether the first and second source satisfy a predetermined genetic at least one criterion relating to social compatibility,
wherein the first and second genetic data is related to normal functions and unrelated to disease or illness.

23. The system of claim 22, further comprising:
a means for generating a first and second coded pattern identifiers;
a means for associating the first and second coded pattern identifiers to the data source; and
a means for updating the data source to include the first and second coded pattern identifiers.

24. The system of claim 22, further comprising the means for enabling a user to display plurality of images as a screen saver, said screen saver cycling through said plurality of images.

25. A computer program product comprising a computer readable medium having computer readable code embodied therein, the computer readable code, when executed, causing a computer to implement a method for determining compatibility of a plurality of genetic data from at least one of a plurality of sources, wherein the genetic data are unrelated to disease or illness genotypes, the method comprising:
receiving a first genetic data from a first source and a second genetic data from a second source;
comparing the first and second genetic data to a data source, wherein the data source comprises a plurality of socially relevant genes;
determining whether the first and second genetic data comprise at least one of the plurality of socially relevant genes;
displaying a first image corresponding to the first genetic data and a second image corresponding to the second genetic data;
identifying a plurality of first and second identifiers from the first and second genetic data, wherein the first and second identifiers indicate a plurality of social traits; and
comparing the first and second identifiers to determine whether the first and second source satisfy a predetermined at least one criterion relating to social compatibility,
wherein the first and second genetic data is related to normal functions and unrelated to disease or illness.

26. The computer program product of claim 25, wherein the implemented method includes:
combining the compared first and second identifiers;
transmitting the combined first and second identifiers to the second source; and
generating a third image corresponding to the combined first and second identifiers.

27. The computer program product of claim 25, wherein the implemented method includes:
generating a first and second coded pattern identifiers; and
associating the first and second coded pattern identifiers to a data source, wherein the data source comprises a plurality of socially relevant genetic data.

28. A computer program product comprising a computer readable medium having computer readable code embodied therein, the computer readable code, when executed, causing a computer to implement a method for determining compatibility of a plurality of genetic data from at least one of a plurality of sources, wherein the genetic data are unrelated to disease or illness genotypes, the method comprising:
receiving a plurality of genetic data from a plurality of sources;
determining whether the plurality of genetic data comprises at least one of the plurality of socially relevant genes;
identifying a plurality of identifiers from the plurality of genetic data, wherein the identifiers indicate a plurality of social traits;
comparing the identifiers to determine whether at least one of the plurality of sources satisfies a predetermined genetic compatibility;
generating an image corresponding to the compared identifiers, wherein the compared identifiers corresponding to at least one of the plurality of sources satisfy at least one criterion relating to social compatibility; and
transmitting the image to at least one of the plurality of compatible sources,
wherein the plurality of genetic data is related to normal functions and unrelated to disease or illness.

29. The computer program product of claim 28, wherein the implemented method includes:
determining proximity of at least one of the plurality of the compatible source; and
generating a signal to indicate the proximity of at least one of the plurality of the compatible source.

30. The computer program product of claim 29, wherein the implemented method includes generating a signal to indicate a level of genetic compatibility of at least one of the plurality of the compatible source.
